(19) United States
(12) Patent Application Publication
(10) Pub. No.: US 2018/0056063 A1
(43) Pub. Date: Mar. 1, 2018

(54) CLOSED LOOP SYSTEM FOR MODULATING GASTRIC ACTIVITY AND USE THEREOF

(71) Applicant: New York Institute of Technology, Old Westbury, NY (US)

(72) Inventors: Aydin FARAJIDAVAR, Old Westbury, NY (US); Leo CHENG, Auckland (NZ); Greg O'GRADY, Auckland (NZ)

(21) Appl. No.: 15/686,527

(22) Filed: Aug. 25, 2017

Related U.S. Application Data
(60) Provisional application No. 62/379,783, filed on Aug. 26, 2016.

(51) Int. Cl. A61N 1/36; A61N 1/05; A61B 5/0492; A61B 5/00

(52) U.S. Cl. CPC A61N 1/36007; A61N 1/0509; A61B 5/6871; A61B 5/4836; A61B 5/0492

(57) ABSTRACT

The invention relates to a closed loop system which is used in modulating gastric activity. By appropriate deployment of the components of the system, one can monitor and modulate “slow waves” in the gastrointestinal system of a subject.

### RELATED APPLICATIONS

This application claims priority of provisional application 62/379,783, filed Aug. 26, 2016, and incorporated by reference in its entirety.

### FIELD OF THE INVENTION

This invention relates to a system and apparatus useful inter alia in treatment of gastrointestinal disorders, in enhancing post gastric surgery and recovery, and in disrupting stomach motility to reduce gastric emptying.

### BACKGROUND AND PRIOR ART

Gastric peristalsis is initiated, and coordinated, by underlying bioelectrical activity, called “slow waves.” These are generated and propagated by specialized cells found in the gastrointestinal (GI) tract wall (intestinal cells of Cajal). See, e.g., Huizinga, et al., Am. J. Physiol. Gastrointest. Liver Physiol., 296:1-8 (2009). (All references cited herein are incorporated by reference in their entirety).

Abnormalities in this activity, i.e., slow wave patterns, or dysrythmias, have been associated with gastric dysmotility in gastric disorders including, but not limited to, functional dyspepsia and gastroparesis, referred to as “GP” hereafter, as well as chronic unexplained nausea and vomiting, functional dyspepsia, gastro-esophageal reflux disease and post-operative dysmotility. See, Lin, et al., Neurogastroenterol. Motil., 22:56-61 (2010); Leahy, et al., Am. J. Gastroenterol., 94(4):1023-1028 (1999). Dysrhythmias may also occur after gastric operations such as gastric bypass, pancreaticoduodenectomy, gastrectomy and partial gastrectomy. If dysrhythmias persist long after the surgery, they may cause chronic dysmotility See, Berry et al., Obes. Surg., 27:1929-1937 (2017). See O'Grady, et al., Clin. Exp. Pharmacol Physio 41: 854-862 (2014).

GP, which is also referred to as “delayed gastric emptying,” affects more than 1.5 million people in the United States alone, and it is a severe problem in about 100,000 people. It is a condition that is especially common among sufferers of Type I diabetes, and approximately 20% of Type I diabetes sufferers also suffer from this problem. As is implied in its name, food in the stomach either stops moving or moves more slowly to the small intestine than is normal. The mortality rate for GP sufferers is about 5-10%, with some believing the rates are higher for Type I diabetes sufferers. Keith-Ferris, et al., (2003). (http://www.digestivedistress.com/sites/default/files/pdf/White_Paper_%20on_GI_moitility_diseases_GPDA_submis.pdf)

Functional dyspepsia (FD), is a chronic disorder of sensation and movement in the upper digestive tract. It is likely that multiple mechanisms, including abnormal gastric emptying, visceral hypersensitivity, impaired gastric accommodation and CNS factors are involved, and result in stomach pain, bloating and gas. FD is also associated with disorders such as gastrointestinal reflux disease (GERD), and gastroparesis. It is a very common condition throughout the world. Jeong, et al., World J. Gastroenterol., 14:6388-6394 (2008); Piessevaux. et al., Neurogastroenterol. Motil., 21:378-388 (2009). Up to 29% of the U.S. population is affected by FD. Shahib, et al., Am. J. Gastroenterol., 99:2210-2216 (2004).

The diagnosis of these, and other gastrointestinal disorders, can be accomplished via, e.g., physical examination, medical history, blood tests, a radioisotope gastric emptying scan, upper GI endoscopies, upper GI X-ray studies using barium, ultrasound, and the “Smart Pill.” NDDIC: http://digestive.niddk.nih.gov/ddiseases/pubs/gastroparesis#14; Parkman, et al., Gastroenterology, 127:1592-1622 (2004); Waseem, et al., PV World J. Gastroenterol., 7:15(1):25-37 (2009). These diagnostic strategies function via exclusion, rather than analyzing basic mechanisms. None allow for real time continuous monitoring of slow wave dysrythmias, which contributes to the symptoms and the pathology.

Most approaches to treatment of these disorders involves an implantable pulse generator, or “IPG.” These devices, which are similar to cardiac pacemakers, stimulate the stomach by pacing (defined infra) gastric myoelectrical activity, i.e., slow waves circumferentially and distally towards the pylorus, increasing amplitude and velocity of propagation. This gastric electrical stimulation (or GES), has potential to alter gastric neuromuscular activity and perception of symptoms, and has shown efficacy in, inter alia, accelerating gastric emptying, reducing both nausea and vomiting in syndromes such as GP. The technique has also been used to blunt appetites and to reduce food intake in the morbidly obese.

In addition to the GES effects discussed herein, some evidence suggests the approach increases the pressure on the lower esophageal sphincter in both anesthetized and conscious dogs. Xing, et al., Dig. Dis. Sci., 8:1481-1487 (2005); Xing, et al. Obes. Surg., 15:1321-1327 (2005). Hence, it is a candidate for treating GERD, but further studies are needed.

Generally, two methods of GES are used for potential therapeutic effects. These are short pulses (high frequency/low energy) and long pulses (low frequency/high energy; i.e. ‘pacing’). Both methods require implantation of an IPG and electrodes either at laparotomy or laparoscopically, on the gastric serosa, or implantation via minimally-invasive methods such as by endoscopic attachment without implantation of the IPG.

Recent developments have led to the use of mini laparotomy and laparoscopy, in place of classic laparotomy. In laparoscopy, e.g., electrodes are inserted into the gastric wall and a pulse generator is inserted in the subcutaneous pocket, without increasing postoperative complications.

The technique of high resolution (HR) mapping has been widely used to elucidate electrophysiological properties of muscles, the heart, the brain, and other organs. Electrocardiography, for example, has provided valuable information about normal and dysrhythmic cardiac electrical behavior. HR mapping has also been used to understand the mechanisms underlying the origin and dysrythmias of gastric slow waves. It has revealed complex focal activities and waveform reentry patterns not apparent in earlier studies, using fewer electrodes. The art regards the detailed characterization of slow wave dysrhythmias as a priority, as it is believed they underlie conditions such as GP, and post operative items. See, e.g., Lammers, et al., Gastroenterology, 135:1601-1611 (2008); Chen, et al., Dig. Dis. Sci., 41:1538-1545 (1996); Vittal, et al., Nat. Clin. Pract. Gastroenterol. Hepatol., 4:336-346 (2007); Lin, et al., Am. J. Physiol. Gastrointest. Liver Physiol., 280-G1370-1375 (2001); Du. et al., Ann. Biomed Eng., 37(4):839-846 (2009); Zhu, et al., Neurogastroenterol. Motil, 17:628 (2005).

A survey of the patent literature shows material useful as background information but not relevant to the invention described infra. U.S. Pat. No. 5,690,691 to Chen et al. describes an open loop system for GI stimulation. U.S. Pat. No. 6,132,372 to Essen-Moller describes a device for recording GI activity that is neither wireless nor implantable. U.S. Pat. No. 6,411,842 to Cigaina et al. describes a device useful for recording GI electrophysiologic activity but lacks the teachings of wireless transmission, managing or motility. U.S. Pat. No. 7,177,693 to Starkenbaum teaches GI stimulation but not a close loop system, wireless power transmission or recording of data.

U.S. Pat. Nos. 7,292,889; 7,343,201; 7,720,539; 7,363,084; 7,775,967; 7,941,221; 8,364,269; and 8,417,342 all lack one or more of the features discussed supra. To the same end, attention is drawn to publisher of U.S. Patent Applications 2014/0058282 and 2013/0035576 to O'Grady et al., which lack the teachings of a closed loop system or GI stimulation.

### SUMMARY OF THE INVENTION

The invention relates to a closed loop system useful in treating GI disorders by changing the bioelectric patterns (slow waves), which are involved in GI movement such as peristalsis.

### BRIEF DESCRIPTION OF THE FIGURES

FIG. 1 shows a 64 channel, flexible and implantable electrode which can be used in the invention.

FIG. 2 shows deployment of electrodes in one embodiment of the invention.

FIG. 3 shows an embodiment of the recording devices of the invention.

FIGS. 4a and 4b show a transducer in accordance with the invention.

FIGS. 5a and 5b depict another embodiment of the transducer of the invention, with a computer model of 3d structure.

FIG. 6 is a schematic view of a signal conditioning circuit.

FIG. 7 shows a capacitor used in embodiments of the invention.

FIGS. 8a and 8b show, respectively, rings positioned in the stomach in accordance with an embodiment of the invention, and longitudinal mobility signal printouts.

FIG. 9 displays a simplified finite state machine model of the invention.

FIGS. 10a, 10b, and 10c show signals from an embodiment using 8 rings as discussed supra, showing normal, bradygastric, and tachygastric activity.

FIG. 11 shows results of an experiment to deliver high energy pulses in vivo, using the invention.

FIG. 12 shows that the invention covers the modulation of the stomach slow wave activity.

FIG. 13 presents a block diagram of one embodiment of the invention.

### DETAILED DESCRIPTION OF PREFERRED EMBODIMENTS

Expressed broadly, the invention is a closed loop system useful for the management of gastrointestinal (“GI”) disorders, and methods for using this system.

The invention, as will be elaborated upon infra, allows for the modulation of GI electrical activity as well as other activity (e.g., peristalsis), from a plurality of different channels. Further, the system permits delivery of various forms of electrical stimulation, as necessary and/or desirable, also through multiple channels. This implies that the system permits “real time” recordal and analysis of GI signals, such that the appropriate electrical stimulation can be transmitted to the stomach or other parts of the GI system.

In an embodiment of the invention, if more comprehensive analysis of signals is desired, the recorded signals may be transmitted wirelessly to, e.g., a computer for analysis.

As will be elaborated upon further, infra portions of the system are implanted and others can be, but need not be, placed outside of the body. These portions can be portable, or wearable, and the wearable or portable portion is connected to the implanted electrodes, e.g., in the stomach, again as discussed infra. The following discusses preferred embodiments of the invention.

In a completely implantable embodiment, the system comprises a plurality of multichannel electrodes, which record and/or deliver electrical signals from and to the stomach. These electrodes are implanted in the serosa, or mucosa of the stomach, and connect directly to an implantable control unit described herein. These electrodes are used in all embodiments of the invention.

The implantable control unit, or “IU” has a single microcontroller, and the IU comprises each of:
(i) means for managing power;
(ii) a recordal means;
(iii) an electrical stimulation means, and
(iv) a signal analysis means.

The skilled artisan will be familiar with various embodiments of each of (i) to (iv). For example, (i) can include a rechargeable battery, a coil, a battery charger, a DC to DC converter, and a low voltage detector. Recordal means (ii) can include one or more amplifiers and/or filters, an analog to digital converter (“ADC”), a multiplexer, and a wireless receiver. The electrical stimulator (iii) can include a digital to analog converter (“DAC”), a current transceiver source and a demultiplexer. Finally, item (iv) includes a field programmable gate array or a digital signal processing integrated circuit. All of (i) to (iv) utilize the same microcontroller.

An additional possible component of the implantable system is a wearable or portable unit, which receives signals from the implanted electrodes and can relay them to the stationary unit, discussed infra. This wearable/portable unit can also receive stimulation commands from the stationary unit, which are relayed to the IU and electrodes. It can also recharge the IU battery wirelessly. In all embodiments, it comprises a power amplifier, a microcontroller, a wireless transceiver, battery, and a coil.

The stationary unit, referred to supra, comprises a transceiver in communication with the wearable unit, and a graphic interface which runs on a computer.

In the wearable or portable embodiment, the same multichannel electrodes referred to supra for the implantable embodiment are used. These are connected, via, e.g., wires, to the wearable/portable unit (WU) described infra. The WU is external to the patient's body, so the wire passes through, e.g., a natural orifice such as the nasal cavity, or traverses the abdomen.

The WU does not need to be recharged wirelessly. Hence, it comprises a replaceable battery, or other replaceable power source. The stationary unit described supra is also part of this embodiment. It is otherwise identical to the IU, supra.

In operation, the invention delivers closed loop therapy for GI disorders including, but not limited to, gastroparesis, functional dyspysia, chronic nausea, and vomiting, as well as in connection with other therapies to enhance recovery after, e.g., gastric sleeve or the other GI pathologies and treatments discussed.

In all uses, the invention records GI electric activity and motility, analyzes these data, detects abnormal activity, and delivers electrical stimulation in order to restore normal function.

In its broadest embodiment, the invention requires at least three recording sites (via implantable electrodes) to receive electrical gastric activity in order to analyze gastric slow waves. It is best that these recording sites are located in a triangular configuration, but this is not required. Increasing the number of the electrode recording sites improves the resolution of analysis; hence, better detection of the slow wave speed and direction.

Also in its broadest embodiment, the invention requires at least two electrodes to deliver electrical stimulation to the stomach. One delivery electrode serves as the source and the other one as the sink. By increasing the number of electrodes, one can improve control over stomach motility (finer control); however, in this case, only the number of source electrodes needs to be increased, and one sink electrode is enough. More electrodes improve signal resolution, but the number of electrodes must be balanced against space limitations of, e.g., the stomach. These may be placed anywhere from 5 mm to 5 cm apart, so as to close the electric current loop. Stimulation takes the form of bipolar pulses that last for 100-700 ms, or 3-5 ms at 30-40 Hz. The amplitude of the pulses can vary from 1 to 10 ms. When these pulses are applied at a frequency similar to that of the stomach, they “pace” the stomach. At higher frequency (e.g., two times higher), they suppress motility.

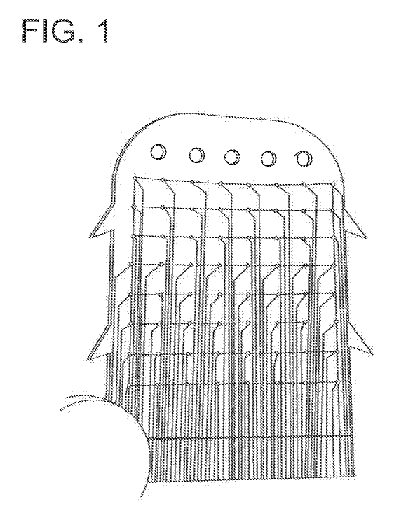

The electrodes, when implanted, may be implanted in ring formation, as discussed infra, or in an array patch as is shown in FIG. 1. It should be noted that the electrode depicted comprises four “fins,” and five holes, which facilitate anchoring for suturing and growth of issue. These, however, are not necessary.

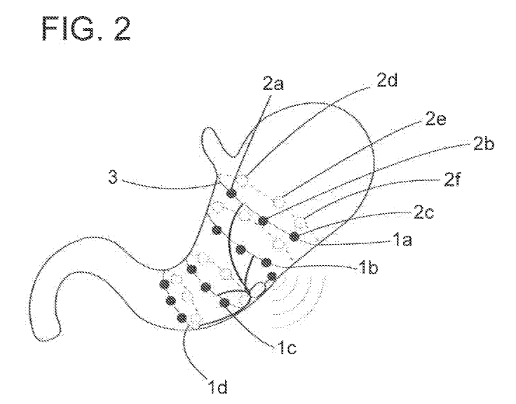

Referring to FIG. 2, an electrode array is shown positioned in a stomach. As can be seen, the array comprises a plurality of discrete rings, “1a” through “1d,” each of which joins a plurality of recording sites “2a-2f” together.

The recording sites are connected to each other by a ribbon or other linear connecting means, “3” in FIG. 2 preferably made of nitino alloy, but which can be made of any inert, biocompatible material. The rings “1a-1d” are flexible in the radial direction, such that they can expand or contract with the movement of the stomach. Since the rings can expand and contract, they can conform with the natural motility of the stomach.

The composition of the rings, as will be elaborated upon infra, is such that they can be attached to the mucosal or serosal surface of the stomach.

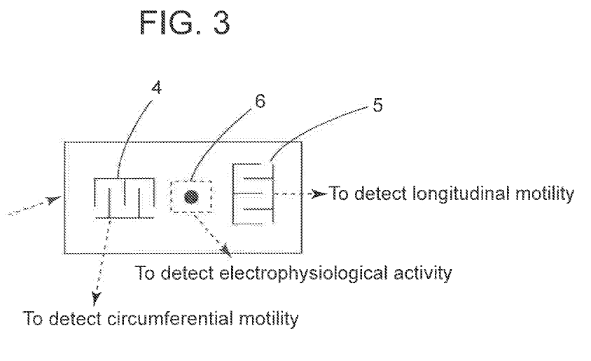

As noted supra, each ring comprises a plurality of recording sites. FIG. 3 presents a more detailed view of each of the recording sites.

Each recording site can consist of up to three sensor types “4,” “5,” and “6.” Sensors “4” and “6” are interdigital transducers and, as can be seen in FIG. 3, they are placed in different directions relative to each other. These interdigital transducers detect the circumferential and longitudinal motility of the stomach.

Item “6” is a metal pad, which is preferably circular. This sensor detects electrophysiological activity. It can be made of any biocompatible metal such as gold, platinum, silver, or alloys such as stainless steel, titanium, etc. Each pad represented by “5” is from about 0.3 to about 1 mm in diameter, and from about 0.1-about 0.3 mm thick.

The electrodes are preferably made of a polyamide substrate, but any inert, physiologically acceptable polymer or composite can be used.

The electrodes (shown in FIG. 2 as 2a-2d) themselves may range from about 2 cm² to about 20 cm² in size, and the shape may vary (e.g., they can be circular, rectangular, triangular, etc.). The size will depend on how many sensors are used in each ring.

While difficult to see in FIG. 2, the tips on “6” are raised, which facilitates contact and anchoring of the sensor pad to the stomach.

As noted supra, sensors “4” and “5” serve as interdigital transducers.

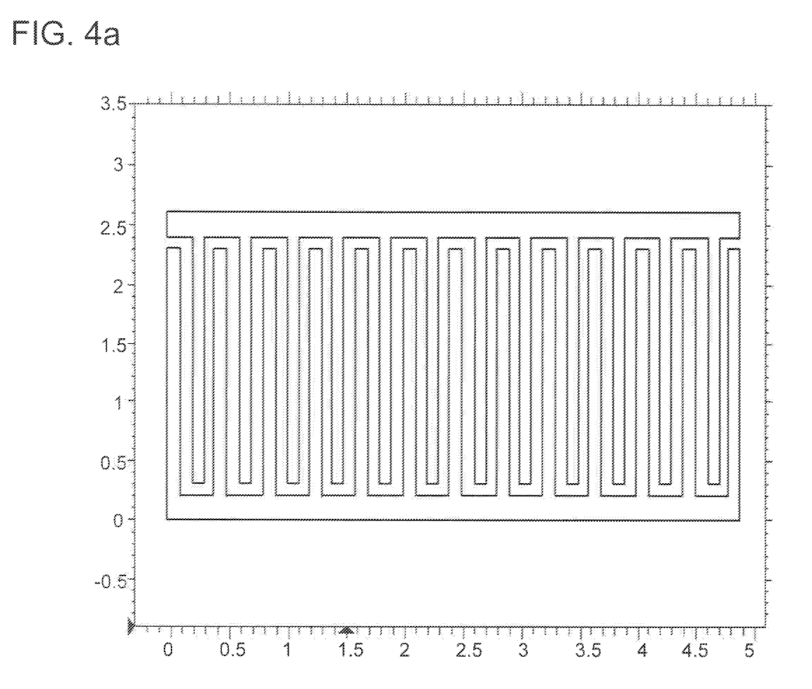

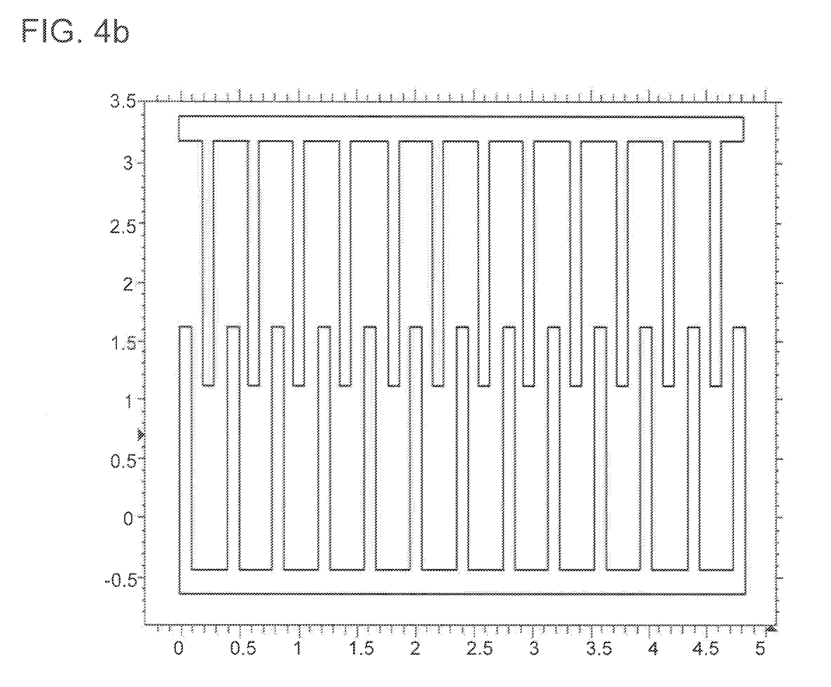

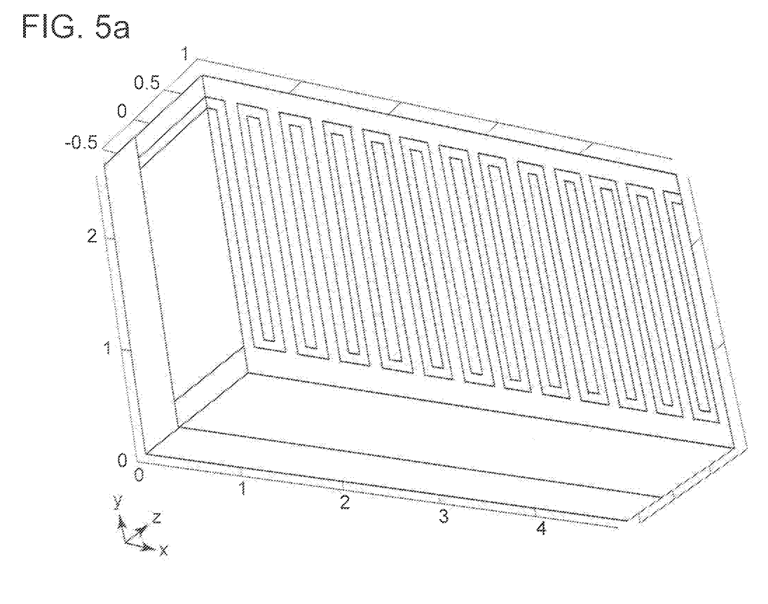

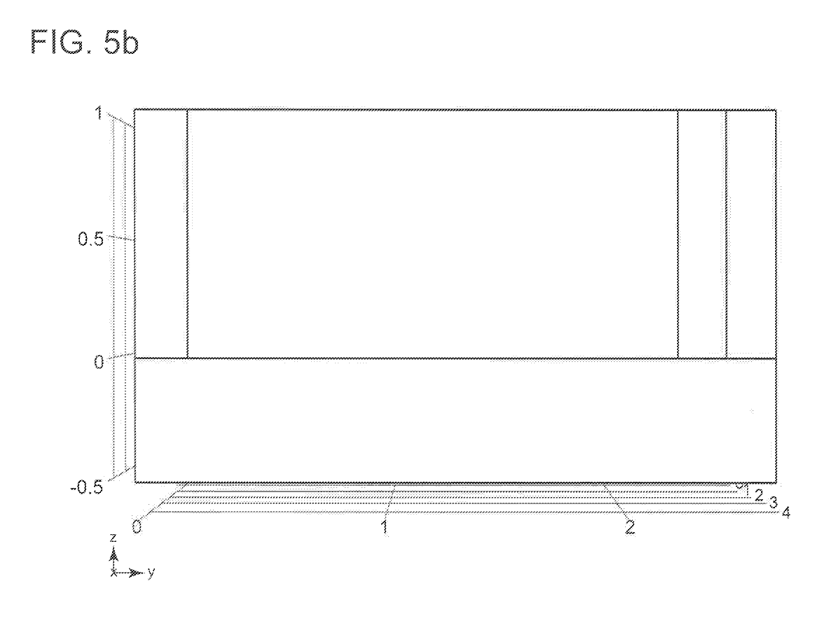

Referring to FIGS. 4 and 5, they consist of two, comb shaped structures made of the same material as the material of the tips discussed supra. The two comb shaped structures face each other, and each is connected to a different voltage level, thus creating a capacitor. The transducers are deposited in polyamide or another polymer, as described supra.

When the stomach moves, the surface area between the two comb structures changes, as is seen by comparing FIGS. 4a and 4b, and the capacity of the structures change. The mobility of the comb structures is limited to from about 5 mm to about 30 mm, in both circumferential and longitudinal directions. FIG. 5 shows a computer model of the 3D structure of a transducer in accordance with the invention. In the model, the following parameters were used:
W: 0.1 mm (width of digits)
G: 0.1 mm (distance between digits on opposite sides)
X: 0.4 mm distance between digits on same side)
L: 30.2 mm (length of each digit)
Width: 50 mm (width of the sensor)

Up to 2.8 cm of motion could be detected.

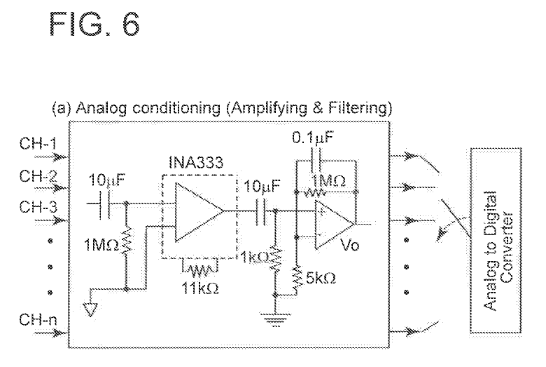

When electrophysiolical signals pass through the recording pads discussed supra, they stream to signal conditioning circuitry, an example of which is shown in FIG. 6.

One sees, in this figure, a low power precision instrumentation amplifier “9,” and a zero drift operational amplifier “10.” The signals from the recording pads pass through a high pass filter (e.g., 0.01 Hz), to “9,” being amplified by a factor of “10.” The signal then passes through a second order band pass filter and amplifier (set at, e.g., 0.01-1.6 Hz), which amplifies the signal by 200.

See, e.g., Paskarandanadivel, et al., Neuroenterol & Motil, 27(4): 580-585 (2015); J. Physiol Mear, 33(6): N27-37 (2015); and Farajidavar, et al., 39, of which are incorporated by reference, for information on successful manufacture of such signal conditioning means.

Filtered signals are sampled, digitalized by an analog to digital converter (ADC), and processed by a field programmable gate array, or “FPGA.” Both structures are known to the skilled artisan.

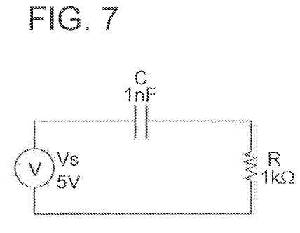

Stomach motility is detected via changes in the capacity of the aforementioned interdigital sensors. While the art is familiar with many ways to measure this parameter, one embodiment, shown in FIG. 7, models the sensor as a capacitor. We can add a resistor “10” in series to this capacitor. The capacitor is charged fully every 5 seconds, using a voltage controlled voltage source “11,” with the current flowing in the circuit (I) being measured. Ohm's Law:

V=ZI,

with Z=−j/Cω, and ω serving as frequency, defines the voltage over the sensor. When V is known, and I is measured, capacitor (c) changes are easily calculable.

### Example 1

An “in silica” ICC network model was prepared, based upon Finite State Machine theory. In this model, three states are used; (i) an initial state, representing rest potential; (ii) a passive state, representing a non-refractory period, and (iii) an active state containing slow wave potential.

These considerations led to a network of ICCs, containing 8 rings as shown in FIG. 2. These rings are placed along the great curvature of the stomach, from corpus to antrum, with two, four, and two rings for the pacemaker, corpus, and antrum rings. Anywhere from 12-30 virtual ICCs (interstitial cells of Cajal) are contained in each ring, based upon its location within the stomach. Sequential activation of each of the ICCs in super state. This induces activation of other rings, in antegrade sequence.

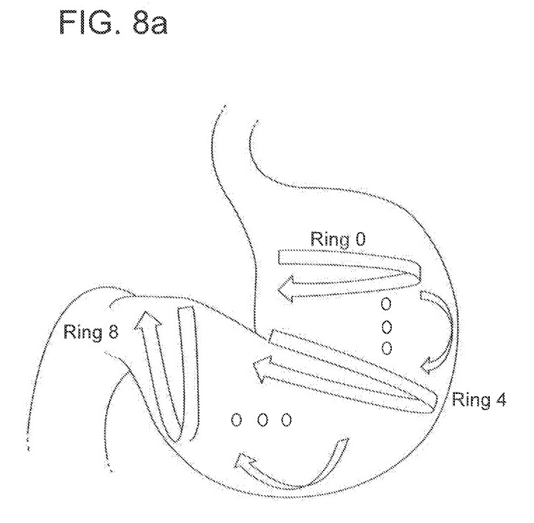

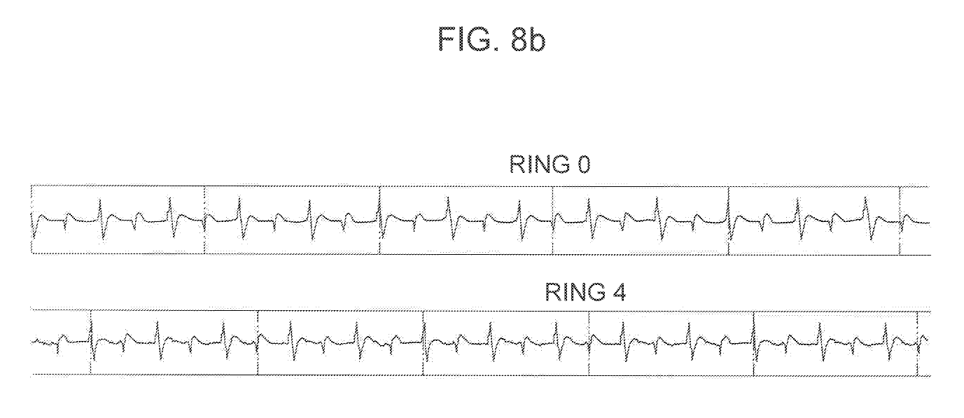

FIGS. 8a and 8b show, respectively, the rings positioned in the stomach (8a) as well as circumference longitudinal motility (8b) signals.

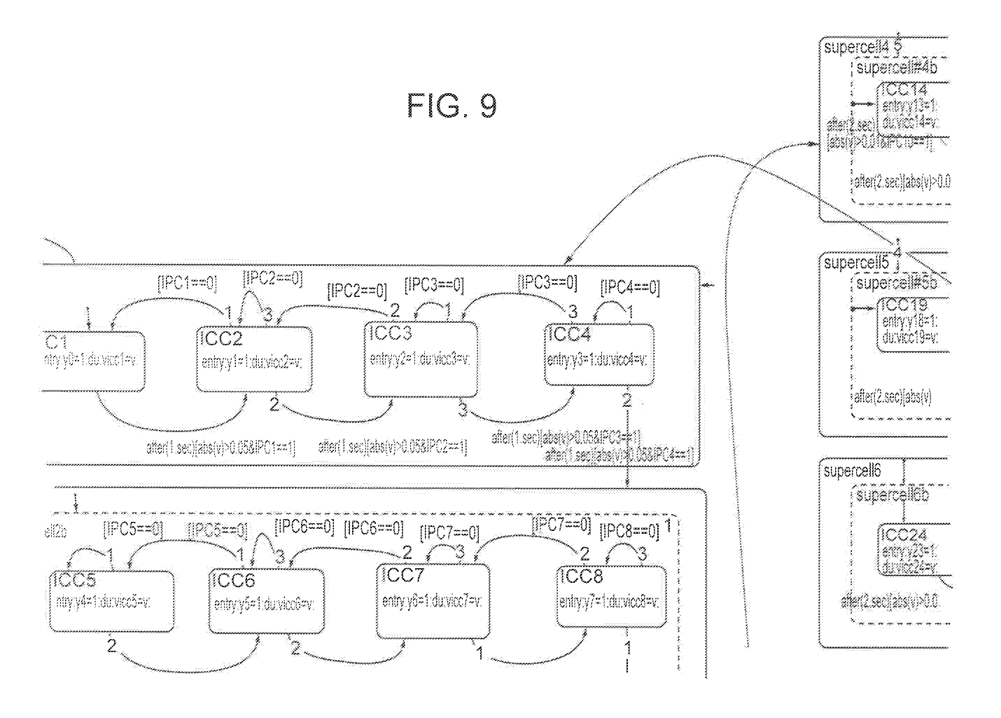

In FIG. 9, one sees a simplified finite state machine (FSM) model, with eight super states. Each superstate includes 4-5 sub-states, wherein a more realistic model can include up to 30 or more substates.

The model, which is art recognized, simulates normal gastric rhythmically, and also models disrhythmic patterns and frequencies, such as occur in bradygastria, and tachygastria. When this model uses an input basis on a mathematical function akin to a single ICC activation, such as a combination of an upstroke and a plateau phase, the model responds with normal antegrade activation, at 3 cpm. Propagation velocity matches expected physiological values (i.e., 8, 3, and 5.7 mms for the pacemaker, corpus, and antrum regions). Decomposing the same input function into high or low frequency signals using a chebyshev filter, permits simulation of tachygastric (5 cpm) and bradygastric (2 cpm) conditions.

Additionally, by applying external pulse trains at 3 cpm to the 4^{th} ring, a pacing application is demonstrated.

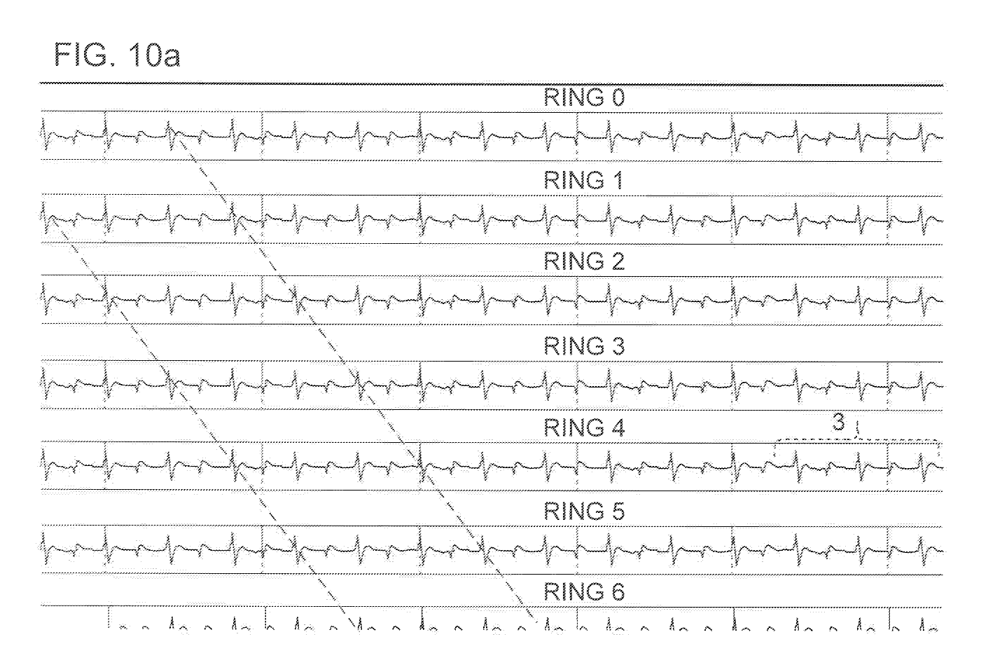

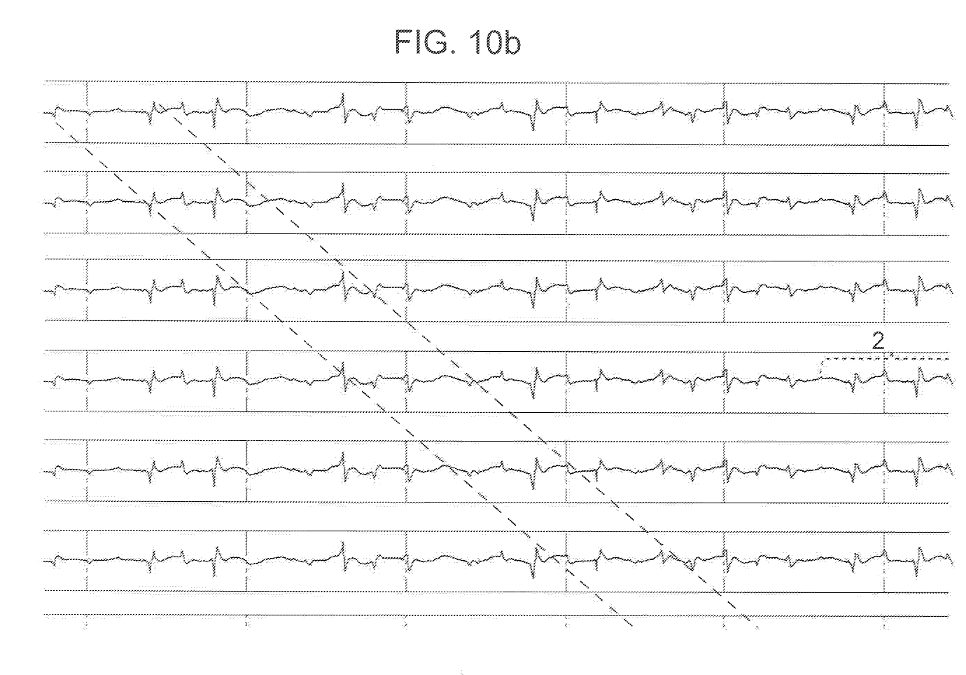

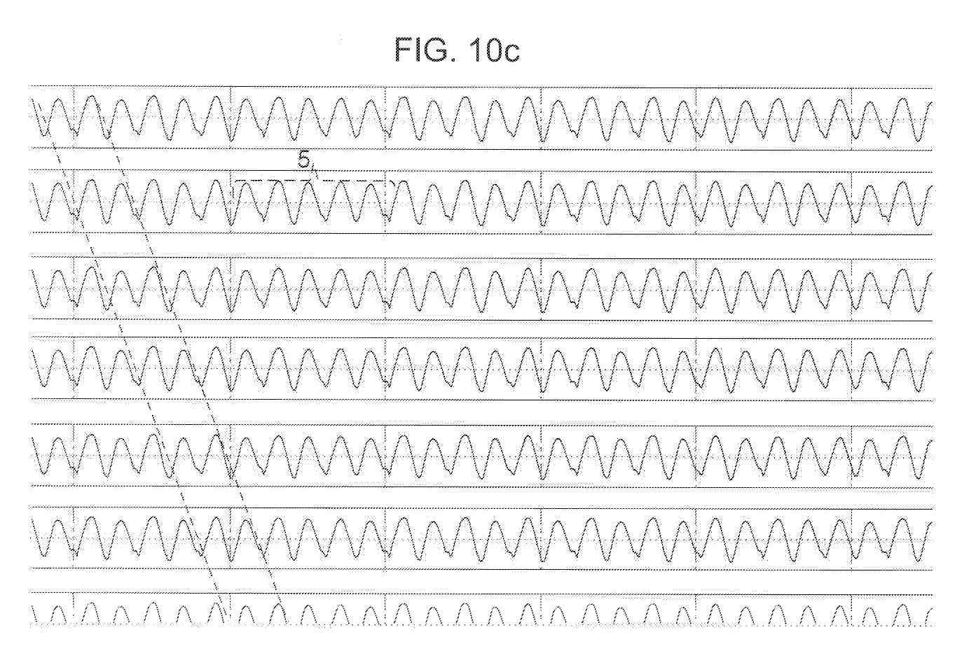

FIGS. 10a, 10b, and 10c show the simulation model in normal, bradygastric, and dysrhythmic activities.

This model, or a similar model, can be included as part of the signal analysis means, supra.

### Example 2

As was elaborated supra, laparoscopic sleeve gastroectomy (LSG) is becoming a common method to treat obesity. This procedure involves the excision of the gastric pacemaker, which can lead to gastric dysrhythmia, leading in turn to post operative food intolerance, nausea, and vomiting.

In these experiments, high resolution electrical mapping was carried out, so as to define the impact of LSG on gastric slow wave activity.

Electrode array patches were deployed laparoscopically, using flexible printed circuits 8-12 cm², containing 64-96 electrodes, in eight patients.

Slow wave activity was quantified using data on propagation patterns, frequency, velocity and amplitude.

One patient was mapped after LSG, and before gastric bypass.

The results indicated that all patients showed grossly abnormal slow wave pacemaking after sleeve gastrectomy. To elaborate, 50% showed absence of activity, while 50% shows a distal ectopic pacemaker.

One patient with chronic nausea, food tolerance, and discoordinated motility, 6 months after LSG was also mapped. The patient exhibited a stable ectopic pacemaker in the gastric antrum, and rapid retrograde propagation at mean velocity 12.5 mm/s.

### Example 3

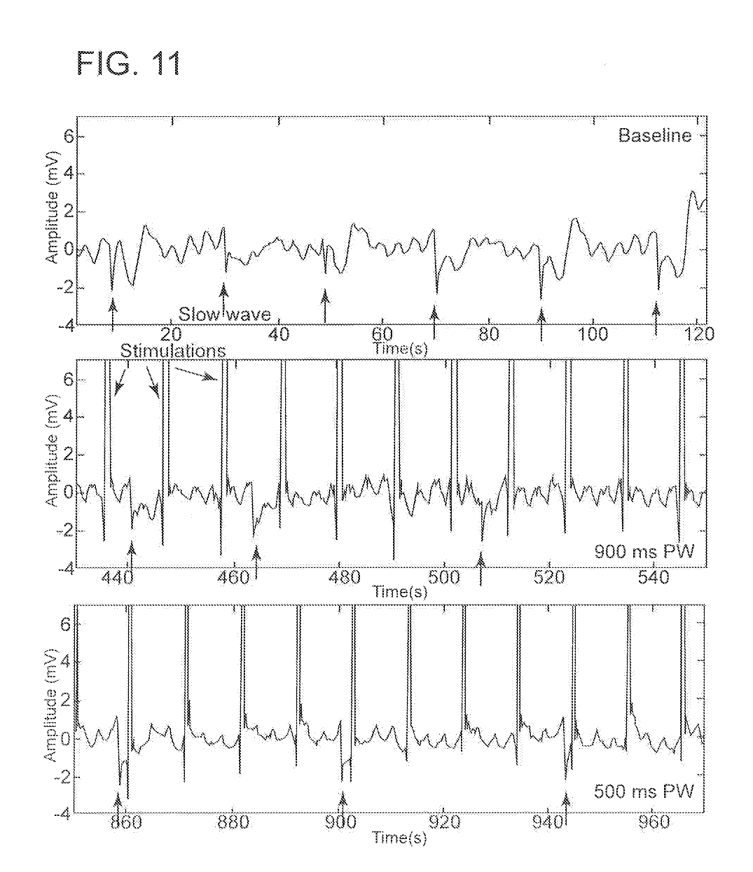

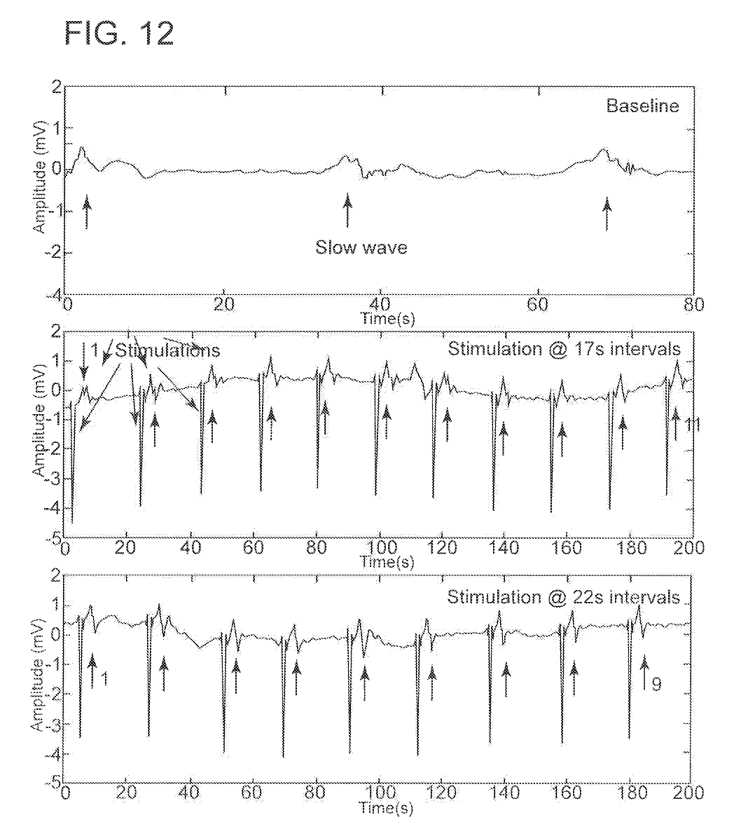

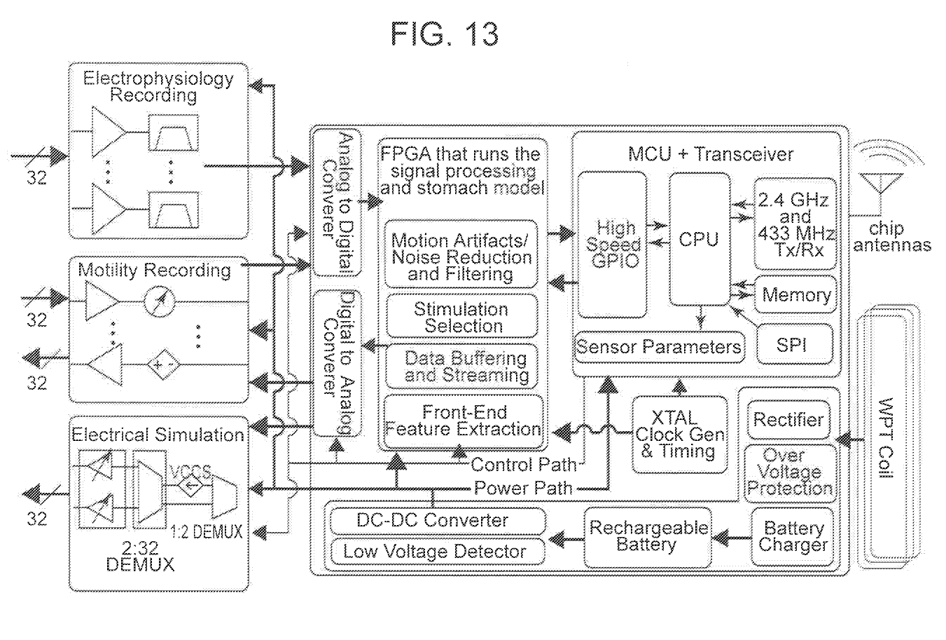

This example tested the ability of the system to modulate stomach motility and either decrease or increase its activity. A swine model was used, in which an electrode array was placed on the mid-corpus to record slow wave activity, and the stimulator electrodes were inserted into the gastric wall adjacent to the proximal edge of the array. Two different experiments were carried out. In experiment #1 stimulation was applied on average for 150 s in the pig at 8 mA, at 10 s intervals with various pulse widths: 500 ms, 900 ms, 500 ms. In experiment #2 stimulation was applied to the pig at 5 mA, at 16-20 s intervals, and pulse width variance of from 500-900 ms. The results are shown in FIGS. 11 and 12, and show successful tissue delivery of high-energy electrical pulses. The system could modulate and decrease slow wave initiation, pattern and frequency (FIG. 11), and successfully entrain slow wave activity. Stomach activity was modulated at different paces (FIG. 12).

The foregoing disclosure describes embodiments of the invention, which is a closed loop system and apparatus useful in regulating gastrointestinal motility, and methods for using this apparatus.

The system/apparatus comprises at least 3 implantable electrodes for recording electrical signals produced, e.g., in the stomach. While more recording electrodes can be used, they are not required.

The system/apparatus also comprises at least two further implantable electrodes, which are capable of delivering electrical stimulation to the tissue or organ in which they are implanted. One of these two “stimulatory” electrodes functions as a source for electrical stimulation, the other as a sink. As with the recording electrodes discussed supra, more than two stimulatory electrodes can be used. Only one “sink” electrode is ever required. Sometimes, these stimulatory electrodes are referred to as “stimulation channels” in the art.

With respect to the recording electrodes, they are preferably positioned in a triangular array or configuration and may be joined, e.g., by a physiologically compatible ribbon or wire, to produce an easily implantable “ring” or “circle,” “ellipse,” etc.

A further feature of the system is a control until, which can be implanted in the subject, at a distance from the electrode arrays discussed supra, or be wearable in, e.g., a vest or belt or other article of clothing, or may simply be portable. In these non-implanted embodiments, the electrodes referred to above are connected to the control unit via, e.g., one or more wires or by any other transmission device which can be passed through a natural body orifice, such as the nares, or via an incision in the abdomen.

When the control unit is implantable, it requires a means for managing power, a recordal means, an electrical stimulation means, and a signal analysis mode. These components are required when the unit is implantable or wearable/portable as well. In the implantable format, there must be a rechargeable battery or other source of power as well.

Optionally, the apparatus/system of the invention can include additional external components for receiving and analyzing data from the unit and electrodes. In operation, the electrodes are implanted in a subject at a point in the GI system requiring attention. Generally, this is the stomach, but other loci are possible. If the control unit is implanted it, too, is placed at the loci close to the electrodes. The electrodes receive the electrical information that is provided by the natural activity of the GI system, and transmit this to the control unit. After analyzing this information, the control unit generates an appropriate electrical stimulation to regulate or alter the motility of the stomach or other component of the GI system. The electrical stimulation can be sent to the implanted electrodes to, e.g., instigate stomach emptying or some other desired goal.

Other embodiments of the invention will be clear to the skilled artisan and need not be repeated here.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

1. A system useful in modulating gastrointestinal electric activity, comprising:
(a) a control unit which comprises:
(i) a microcontroller;
(ii) a means for managing power,
(iii) a recordal means;
(iv) an electrical stimulation means; and
(v) a signal analysis means;
(b) at least three implantable recording electrodes; and
(c) at least two implantable stimulation electrodes, one of which delivers electrical stimulation, and the other of which is a sink.

2. The system of claim 1, wherein said control unit is implantable.

3. The system of claim 2, further comprising an external means for receiving and delivering signals to said control unit.

4. The system of claim 1, wherein said implantable recording electrodes are connected via a physiologically acceptable ribbon.

5. The system of claim 4, wherein said ribbon is made from nitinol ally.

6. The system of claim 1, wherein said recording electrodes comprise interdigital transducers and metal pads.

7. The system of claim 1, wherein said electrodes are made of gold, platinum, silver, stainless steel, or titanium.

8. A method for monitoring electrical activity in a gastrointestinal tract, and delivering electrical stimulation comprising implanting:
(a) at least three recording electrodes;
(b) at least two stimulating electrodes; and
(c) a control unit which comprises each of:
(i) a microcontroller;
(ii) a means for managing power,
(iii) a recordal means;
(iv) an electrical stimulating means; and
(v) a signal analysis means.
in a subject in need of said monitoring, wherein (a), (b), and (c) are positioned so as to provide data from (a) to (c) and (b), and monitoring any electrical activity from said gastrointestinal tract.

9. The method of claim 8 comprising implanting (a), (b), and (c) in the stomach mucosa or serosa.

10. The method of claim 8, further comprising transmitting said signal to a non-implanted signal reception means.

11. A method for monitoring electrical activity in a gastrointestinal tract, comprising implanting:
(a) at least three recording electrodes;
(b) at least two signaling electrodes; and
(c) connecting (a) and (b) to a non-implanted control unit which comprises each of:
(i) a microcontroller
(ii) a means for managing power;
(iii) a recordal means
(iv) an electrical stimulating means; and
(v) a signal analysis means to determine electrical activity in said gastrointestinal tract via said non-implanted control unit.

12. The method of claim 8, further comprising transmitting electrical stimulation to (a), (b), and (c) to modulate gastrointestinal electric activity.

13. The method of claim 11, further comprising transmitting electrical stimulation to (a) and (b) to modulate gastrointestinal electric activity.
